(19) United States
(12) Patent Application Publication
(10) Pub. No.: US 2021/0267454 A1
(43) Pub. Date: Sep. 2, 2021

(54) COMBINATION OF DATA FROM TWO EXTERNAL RESPIRATORY MEASUREMENT SENSORS INTO ONE DATA STREAM

(71) Applicant: MAKANI SCIENCE INC., Irvine, CA (US)

(72) Inventors: Michael Chu, Irvine, CA (US); William E. Saltzstein, Irvine, CA (US)

(21) Appl. No.: 17/184,911

(22) Filed: Feb. 25, 2021

Related U.S. Application Data
(60) Provisional application No. 62/984,055, filed on Mar. 2, 2020.

(51) Int. Cl. A61B 5/00; A61B 5/085

(52) U.S. Cl. CPC A61B 5/0024; A61B 5/7203; A61B 2562/0261; A61B 5/4519; A61B 5/6823; A61B 5/085

(57) ABSTRACT

The present invention is directed to the combination of respiratory data from two or more different sensors in order to generate a combined waveform. The system of the present invention may comprise a set of two strain sensors, each strain sensor comprising a bare sensor and an adhesive layer to apply said sensors to the skin of a patient, one on the chest and one on the abdomen. The system may further comprise a computing device connected to both strain sensors in some form. As the distention of the patient's skin puts strain on the strain sensors, waveforms are transmitted from the sensors to the computing device where both waveforms are combined into a singular waveform.

### CROSS-REFERENCES TO RELATED APPLICATIONS

This application is a non-provisional and claims benefit of U.S. Provisional Application No. 62/984,055 filed Mar. 2, 2020, the specification of which is incorporated herein in its entirety by reference.

### BACKGROUND OF THE INVENTION

The measurement of respiratory activity through the use of external sensors applied to the skin of a patient at the chest or abdomen is well-established as these physical locations on the body have been proven to be the most optimal for measuring a patient's breathing. However, prior systems have struggled to truly process data from both of these physical locations at once. Systems exist that incorporate measurements from multiple sensors applied to the aforementioned bodily locations, but the method of said systems involves selecting a dominant breathing type and only returning data from the sensor associated with the dominant breathing type. The selection of a dominant breathing method is a difficult task with a significant amount of potential error, and furthermore the dominant breathing method may change over the course of respiratory measurement, rendering the original selection useless. Thus, a present need exists for a system that accepts respiratory data from sensors applied to two or more different physical locations on the body and combines said data into a combined waveform.

### FIELD OF THE INVENTION

The present invention is directed to the combination of respiratory data from both the chest and the abdomen using two or more different sensors in order to generate a combined waveform.

### BRIEF SUMMARY OF THE INVENTION

It is an objective of the present invention to provide a system that allows for a combined waveform from respiratory data collected from two or more different strain sensors. A strain sensor is a sensor applied to a patient's skin by an adhesive layer and a waveform is returned corresponding to how much the sensor is strained by the distention of the skin (for instance, when a patient takes a breath) over a period of time.

The system may comprise two strain sensors, each strain sensor comprising a bare sensor and an adhesive layer to apply the sensors to the skin of the patient. One sensor may be applied to the skin of the chest of the patient, and the other may be applied to the skin of the abdomen of the patient. The system may further comprise a computing device that is communicatively coupled to the two strain sensors. The strain sensors may measure a waveform based on the patient's breathing. Said waveforms may then be transmitted to the computing device, where the waveforms are combined into one waveform which is then displayed. The combined waveform may be generated through the use of a simple or complex algorithm. The combined waveform may then be used for subsequent measurements. Without wishing to limit the present invention to any theory or mechanism, it is believed that the technical feature of the present invention advantageously provides for a more accurate and time-efficient method of gathering respiratory data from a patient. This is because data is taken from multiple areas of respiratory activity at the same time, thus allowing for a decrease in the amount of noise in the signal between a sensor and the computing device and for more comprehensive results while minimizing the need for human interaction with the system. Furthermore, the dominant breathing mode of the patient may change over time due to physiological conditions, physical movement, or shifting, which would render prior systems that only measure a dominant breathing method useless. The combined waveform generated by the present invention is unaffected by a shift in the dominant breathing method, increasing the accuracy of the system as a whole.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1a shows both the application of strain sensors on a patient's body and the parts that make up a strain sensor.

FIG. 1b shows a readout from a strain sensor as strain is applied to it to mimic skin distention.

FIG. 1c shows a size comparison of a strain sensor and a coin.

### DETAILED DESCRIPTION OF THE INVENTION

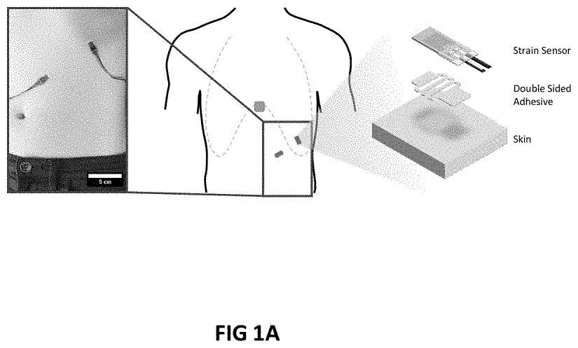

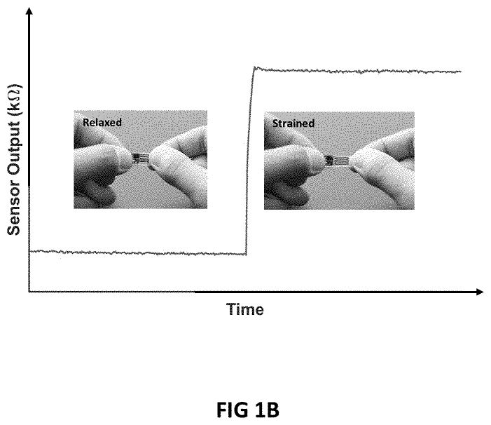

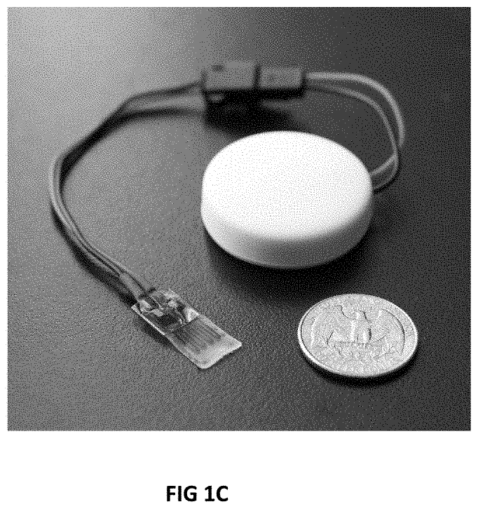

Referring now to FIGS. 1a-c, the present invention features a system for the combination of waveforms from two or more different sensors applied to different bodily locations into a single combined waveform. In some embodiments, the system may comprise two strain sensors. A strain sensor may comprise a bare sensor, and a double-sided adhesive layer such that the bare sensor is applied on top of the adhesive layer and the adhesive layer is then applied on top of a patient's skin. A first strain sensor may be applied to a patient's chest, and a second strain sensor may be applied to a patient's abdomen. In some embodiments, the system may further comprise a plurality of additional sensors attached to a plurality of locations on the body for measuring a plurality of physical signals, wherein the plurality of locations comprises an upper chest region, a lower chest region, a left side of the body, a right side of the body, a back, and/or a lower abdominal region. The system may further comprise a computing device that is communicatively coupled to both of the strain sensors. In some embodiments, the strain sensors may connect to the computing device through a first wire and a second wire, the first wire connecting the first sensor to the computing device and the second wire connecting the second sensor to the computing device. In other embodiments, the strain sensors may connect to the computing device over a wireless connection. In some embodiments, the first sensor may transmit a first physical signal to the computing device based on the distention of the patient's chest as the patient breathes. The second sensor may transmit a second physical signal to the computing device based on the distention of the patient's abdomen as the patient breathes. The computing device may receive signals from both of the strain sensors and generate the combined waveform based on the first physical signal and the second physical signal. The combined waveform may be generated through the use of a simple or complex algorithm applied to the first physical signal and the second physical signal. In some embodiments, the combined waveform may be an average of the first physical signal and the second physical signal. In other embodiments, the combined waveform may be an addition of the first physical signal and the second physical signal. The combined waveform may then be used for subsequent measurements. In some embodiments, the first physical signal and the second physical signal comprise information content at less than 50 Hz. In some embodiments, the first physical signal and the second physical signal comprise information content at 5 Hz to 50 Hz. In some embodiments, the first physical signal and the second physical signal comprise information content at less than 20 Hz. In some embodiments, the first physical signal and the second physical signal comprise information content at 5 Hz to 20 Hz. In some embodiments, processing the plurality of signals into the single data stream may comprise calculating a sum, average, difference, weighted sum, or combination thereof based on the plurality of signals.

In some embodiments, the computing device may further be capable of filtering noise from a plurality of signals. Filtering noise from the plurality of signals comprises utilizing a common mode filter for identifying common mechanical noise and identifying common electromagnetic noise between the first physical signal and the second physical signal. The common noise may be from electromagnetic or mechanical sources. In some embodiments, filtering noise from the plurality of signals may comprise utilizing a filter for identifying and removing noise common to all signals of the plurality of signals, and noise may comprise mechanical noise and electromagnetic noise common to all signals of the plurality of signals. The computing device may utilize a common mode filter in order to filter noise from the plurality of signals. In some embodiments, the computing device is further capable of identifying a physiological status of the patient based on the plurality of signals. The physiological status may be selected from a group comprising obstructed breathing (lungs and abdomen move opposite of each other, a small change in impedance), chest-dominant breathing (a stronger chest waveform), abdomen-dominant breathing (a stronger abdomen waveform), and increased muscle recruitment. If the patient recruits more movement from different locations for breathing, it means their respiration is becoming more difficult. For example, abdominal breathing is normal, while abdominal plus chest wall is normal but may indicate troubled breathing. Recruitment of shoulder and abdominal muscle means there's a problem with respiration.

What is claimed is:

1. A system for combining a plurality of signals collected from sensors placed on an external surface of a body of a patient, the system comprising:
a. a first strain sensor capable of measuring a first physical signal from a first location on the body, wherein the first signal represents an expansion and contraction measurement of the respective location, wherein the first signal includes an electromagnetic noise source and a mechanical noise source at the respective location;
b. a second strain sensor capable of measuring a second physical signal from a second location on the body, wherein the second location is separate from the first location, the second sensor is in a separate orientation from the first sensor, or a combination thereof, wherein the second physical signal represents an expansion and contraction measurement of the respective location, wherein the second physical signal includes an electromagnetic noise source and a mechanical noise source at the respective location; and
c. a computing device capable of processing the plurality of signals comprising the first physical signal and the second physical signal into a single data stream;
wherein the first sensor and the second sensor and any additional sensors are communicatively coupled to the computing device for transmitting the first signal and the second signal respectively.

2. The system of claim 1, wherein the first physical signal and the second physical signal comprise information content at less than 50 Hz.

3. The system of claim 1, wherein the orientation of the first physical signal is orthogonal to the location of the second physical signal.

4. The system of claim 1, wherein the first sensor is placed on the abdominal region of the patient and the second sensor is placed on the thoracic region of the patient.

5. The system of claim 4 further comprising a plurality of additional sensors attached to a plurality of locations on the body for measuring a plurality of physical signals, wherein the plurality of locations comprises an upper chest region, a lower chest region, a left side of the body, a right side of the body, a back, and/or a lower abdominal region.

6. The system of claim 1, wherein the single data stream comprises a waveform.

7. The system of claim 1, wherein the computing device is further capable of filtering noise from the plurality of signals.

8. The system of claim 7, wherein filtering noise from the plurality of signals comprises utilizing a filter for identifying and removing noise common to all signals of the plurality of signals, wherein noise comprises mechanical noise and electromagnetic noise common to all signals of the plurality of signals.

9. The system of claim 1, wherein the computing device is further capable of identifying a physiological status of the patient based on the plurality of signals.

10. The system of claim 9, wherein the physiological status is selected from a group comprising obstructed breathing, chest-dominant breathing, abdomen-dominant breathing, and increased muscle recruitment.

11. The system of claim 1, wherein processing the plurality of signals into the single data stream comprises calculating a sum, average, difference, weighted sum, or combination thereof based on the plurality of signals.
